(19) United States
(12) Patent Application Publication
(10) Pub. No.: US 2008/0118938 A1
(43) Pub. Date: May 22, 2008

(54) Methods and Compositions for the Detection of Protein Folding Disorders

(76) Inventors: Lisbell Estrada, Galveston, TX (US); Claudio Soto, Friendswood, TX (US)

Correspondence Address: FULBRIGHT & JAWORSKI L.L.P., 600 CONGRESS AVE., SUITE 2400, AUSTIN, TX, 78701, US

(21) Appl. No.: 11/851,161

(22) Filed: Sep. 6, 2007

Related U.S. Application Data
(60) Provisional application No. 60/824,639, filed on Sep. 6, 2006.

(51) Int. Cl. G01N 33/53; G01N 33/50; C12Q 1/00

(52) U.S. Cl. 435/7.92; 436/86; 435/4

(57) ABSTRACT

A method is provided for the detection of misfolded proteins in a sample. These methods may be used to diagnose or indicate the potential for developing a disease associated with protein aggregation. In particular a method for serial automated cyclic amplification of a misfolded protein is disclosed.

This application claims priority to U.S. Provisional Patent application Ser. No. 60/824,639 filed Sep. 6, 2006, entitled “Methods and compositions for the detection of protein folding disorders,” which is related to U.S. Utility application Ser. No. 11/407,690 filed Apr. 20, 2006, based on U.S. Provisional Patent application Ser. No. 60/673,302 filed Apr. 20, 2005; and PCT application number PCT/GB01/02584. Each of which is incorporated herein by reference in their entirety.

### BACKGROUND OF THE INVENTION

I. Field of the Invention

The present invention relates generally to diagnostics, pathology, medicine, biochemistry, and cell biology. In particular, the invention provides methods and compositions for the detection of misfolded Aβ proteins in a sample, including the diagnosis of Alzheimer's disease.

II. Background

Alzheimer's disease (AD) is a devastating degenerative disorder of the brain for which there is no effective treatment or pre-clinical diagnosis (Selkoe and Schenk, 2003). A hallmark feature of AD is the misfolding, aggregation, and deposition of amyloid beta protein (Aβ or AB) in cerebral amyloid plaques, which have been proposed as the triggering factor of the pathology (Selkoe, 2000; Soto, 1999; Hardy and Selkoe, 2002). Similar to AD several other neurodegenerative conditions seem to arise from the misfolding and accumulation of protein aggregates in the brain (Soto, 2003), including Parkinson disease, amyotrophic lateral sclerosis, Transmissible spongiform encephalopathies (TSEs), Huntington disease and related polyglutamine disorders. Although the protein involved in the misfolding and aggregation process is different in each disease, the pathological structure in all cases is composed of β-sheet rich amyloid fibrils.

Kinetic studies have shown that protein misfolding and aggregation follows a seeding/nucleation mechanism (Soto, 2003; Harper and Lansbury, 1997), which resembles a crystallization process (FIG. 1). The critical event is the formation of protein oligomers that act as a nucleus to direct further growth of aggregates. Nucleation-dependent polymerization is characterized by a slow lag phase in which a series of unfavorable interactions form an oligomeric nucleus, which then rapidly grows to form larger polymers (FIG. 1) (Soto, 2003, Harper and Lansbury, 1997; Jarrett et al., 1993; Scherzinger et al., 1999; Wood et al., 1999). The lag phase can be minimized or removed by addition of pre-formed nuclei or seeds. At least two intermediates have been identified in the pathway from the native monomeric protein to the fibrillar fully aggregated structure in vitro (Teplow, 1998). The first intermediate is soluble, low-molecular-weight oligomers (dimers to decamers), which have been identified in test-tube experiments, in the conditioned medium of cells that constitutively secrete Aβ, in human cerebrospinal fluid and in human brain homogenate (Kuo et al., 1996; Levine, 1995; Lambert et al., 1998). The second intermediate corresponds to short, flexible, rod-like structures termed protofibrils, which have been studied by electron microscopy, photon correlation spectroscopy, and atomic force microscopy (Walsh et al., 1997). Recent evidence suggests that soluble oligomers and/or protofibrils might be the toxic species in AD and other protein misfolding disorders (Lambert et al., 1998; Gong et al., 2003; Walsh and Selkoe, 2004; Bucciantini et al., 2002).

Currently the diagnosis of AD is based on clinical examination and ruling out other causes of dementia (Nestor et al, 2004). Definitive diagnosis is done post-mortem by brain histological analysis and identification of amyloid plaques and neurofibrillary tangles. No pre-clinical diagnosis is yet possible, and remains one of the highest priorities in the field. Longitudinal studies have shown that the process of protein misfolding and aggregation begin several years or even decades before substantial brain damage and clinical symptoms appear (Mann, 1989; Mann et al., 1990). Therefore, specific and sensitive detection of misfolded and aggregated Aβ protein may lead to a novel diagnosis of AD (Nestor et al., 2004). One of the problems to reach this aim is that misfolded Aβ accumulates exclusively in the brain. Several groups are attempting to develop a non-invasive diagnosis based on imaging of cerebral amyloid plaques (Klunk et al., 2004; Kung et al., 2003). It has been proposed that measurement of Aβ in CSF and blood could be useful for diagnosis of AD (Hampel et al., 2004). However, controversy exists on the utility of these measures for diagnosis, because of the lack of robust and reproducible results. The latter is likely due to the fact that biological fluids contain low quantities of Aβ, which are composed of many different species and distinct aggregation intermediates. An alternative approach might be the specific biochemical detection of some of the precursors of amyloid plaques, in particular soluble Aβ oligomers, which might be circulating in biological fluids decades before the onset of clinical disease. The soluble nature of these species and the data suggesting that they might be the toxic form of Aβ (Lambert et al., 1998; Bucciantini et al., 2002; Gong et al., 2003; Walsh and Selkoe, 2004) makes detection of soluble misfolded Aβ oligomers an interesting target for AD biochemical pre-symptomatic diagnosis. Indeed, the presence of small Aβ aggregates with the capability to act as seeds for Aβ aggregation has been reported in the CSF of humans affected by AD and not in controls (Pitschke et al., 1998). The problem is that quantity of these aggregates is very small and it is difficult to distinguish them from other Aβ species.

### SUMMARY OF THE INVENTION

In recent years much progress has been made in understanding the molecular basis of AD and the development of novel strategies for treatment (Selkoe, 2004). Indeed, several interesting compounds are under clinical evaluation for AD therapy. Considering the low capacity of the brain to regenerate itself, it is very likely that any therapy will have the most potential for producing benefit if treatment is started prior to significant brain damage. Thus, a pre-symptomatic biochemical diagnosis would enable treatment to begin at a time in which little (or no) irreversible damage has yet occurred, e.g., in an asymptomatic subject. A biochemical diagnostic procedure also will be useful to monitor the efficacy of novel treatments and their potential mechanism of action. Such a method is not currently available and there is a need for this type of methodology.

Embodiments of the invention include methods and compositions for diagnosis and/or identification of a misfolded protein in a subject. In certain aspects the methods can be characterized as objective, early, non-invasive, and sensitive biochemical diagnosis or identification of misfolded proteins in a subject. In particular aspects, the method can be used to diagnose or detect misfolded protein associated with Alzheimer's disease or other diseases associated with protein aggregates or aggregation. The detection of misfolded Aβ oligomeric structures in biological fluids can be used in designing a biochemical diagnosis for AD. The methods typically use the functional property of misfolded oligomers to serve as seeds to catalyze the polymerization of monomeric protein (i.e., a substrate protein) as a way to measure their presence in biological fluids. A highly sensitive procedure for the biochemical detection of misfolded proteins such as prions (PrP^{Sc}) has been developed (Saborio et al., 2001; Soto et al., 2002). This technology, termed protein misfolding cyclic amplification (PMCA), reproduce in an accelerated manner the misfolding and aggregation process in vitro, enabling amplification of the misfolded protein marker in the test tube. PMCA is a cyclical process, conceptually analogous to PCR amplification of DNA and consists on cycles composed of two phases. During the first phase the sample containing minute amounts of misfolded oligomers and a large excess of soluble monomeric protein are incubated to induce growth or amplification of misfolded proteins and protein aggregates. In an optional second phase a sample is subjected to ultrasound in order to break down the aggregates, multiplying the number of nuclei. In this way, after each cycle the number of seeds is increased in an exponential fashion. PMCA has been applied to the detection of PrPSc implicated in Transmissible Spongiform Encephalopathies (TSEs) (Saborio et al., 2001) and strikingly to biochemically diagnose the disease during the pre-clinical phase (Soto et al., 2005) and for the first time to detect misfolded proteins in the blood of experimental animals (Castilla et al. 2005). PMCA technology has been modified and adapted for the specific and sensitive detection of misfolded Aβ oligomers, particularly in a pre-symptomatic patient that is suspected of being at risk for the development of AD.

The term “misfolded protein” as used herein is defined as a protein that no longer contains all or part of a structural conformation of the protein as it exists when involved in its typically normal function within a biological system. Typically, a misfolded protein will have a propensity to aggregate or will have a propensity to localize in protein aggregates and is often a non-functional protein.

Embodiments of the invention include methods for detecting a misfolded amyloid β (Aβ) protein in a sample by mixing the sample with a substrate amyloid β (Aβ) protein to make a reaction mix; incubating the reaction mix to enable or provide conditions for the conversion of the substrate amyloid β (Aβ) protein into the misfolded form; and detecting misfolding of the substrate amyloid β (Aβ) protein in the reaction mix. Aspects of the invention include methods that involve amplification of protein or protein fragments by PMCA or serial PMCA (saPMCA). The term PMCA will be used generally to mean either PMCA or saPMCA. PMCA will typically enable high sensitivity detection of proteins or protein fragments associated with protein aggregation and related disease states. In certain embodiments, the method for detecting misfolded proteins involves amplification of the misfolded protein in a sample (which may include serial amplification of the misfolded protein), detection of misfolded protein, and/or inactivation of residual misfolded protein. In certain aspects, PMCA may consist only of incubation of the reaction mix, without the use of sonication. The methods may involve one or more of steps (a), (b), (c), (d) and (e) below:

(a) Mixing a sample with substrate protein to make a reaction mixture (“substrate protein” refers to a preparation of protein or protein fragments that are not present in aggregates of the protein, and they are termed seed-free or low molecular weight form of the protein);

(b) an amplification step comprising:
(i) incubating the reaction mix,
(ii) disrupting the reaction mix,
(iii) repeating steps (b)(i) and (b)(ii) one or more times;

(c) performing serial amplification comprising:
(i) removing a portion of the reaction mix and incubating it with additional substrate protein,
(ii) repeating amplification steps (b), and
(iii) repeating steps (c)(i) and (c)(ii) one or more times;

(d) detecting misfolded or aggregated proteins or protein fragments in the serially amplified reaction mix;

(e) inactivating residual misfolded protein.

Each step is further described below:

(a) Mixing a sample with substrate protein to make a reaction mix. The term “sample” refers to any composition of matter capable of being contaminated with or containing a misfolded protein or protein fragment. For example a sample may comprise a tissue sample from a person suspected of having AD. The term “substrate protein” as used herein refers to a protein or protein fragment that is homologous in all or part to the amino acid sequence of a misfolded or aggregated protein or protein fragment. Typically, the substrate protein or protein fragment has a structural conformation that is typically not identified in biological aggregates of the protein or protein fragment. The substrate protein is generally capable of being converted into a misfolded protein or misfolded protein fragment and may further have a higher propensity for aggregation under typical biological conditions. Thus, “the reaction mix” refers to a composition minimally comprising a sample and substrate protein or protein fragment. In some embodiments, the reaction mix further comprises a “conversion buffer” that is favorable for replication of a misfolded protein. An exemplary conversion buffer may comprise 1× phosphate buffered saline (PBS) with 150 mM additional NaCl, 0.5% TritonX-100 and a protease inhibitor cocktail.

(b) The amplification step involves incubation of the reaction mix under conditions that favor misfolded protein replication (b)(i), followed by disruption of the reaction mix in order to break apart protein aggregates (b)(ii). As used herein the term “disrupting” refers to any method by which proteins aggregates may be disaggregated. Exemplary disaggregation methods include treatment with solvents, modification of pH, temperature, ionic strength, or by physical methods such as sonication or homogenization. These two steps are repeated 1, 2, 3, 4, 5, 10, 20, 30, 40, 50, 100, 200, 300, 400, 500 or more times thereby amplifying the misfolded protein (b)(iii). In certain aspects a portion of the reaction mix can be removed and incubated with additional substrate protein.

(c) The reaction mix from the amplification may be subjected to further amplification and/or serial amplification which greatly enhances replication. In this step a portion of the reaction mix is incubated with additional substrate protein (c)(i) to make a serially amplified reaction mixture. As used herein “additional substrate protein” may be from the same source as the substrate protein used in amplification (a) or it may be from a different source. In some embodiments serial amplification will comprise repeating the steps of amplification (c)(ii) one or more times. In further embodiments, the steps of serial amplification (c)(i) and (c)(ii) are repeated one or more times to further amplify misfolded protein from the sample (c)(iii). By subjecting the sample to sequential serial amplifications the degree of sensitivity is greatly enhanced, allowing detection of fewer than about 10⁵, 10⁴, 10³, 10² or 10 molecules of misfolded proteins or any range derivable therein or even fewer misfolded proteins or fragments thereof.

(d) Misfolded proteins can be detected in the serially amplified reaction mix by both direct and indirect assays known to those of skill in the art. Exemplary methods for detection of misfolded protein in the serially amplified reaction mix are outline below.

(e) Residual misfolded proteins may be inactivated by various methods known to those in the art, such as treatment with a concentrated base or treatment at high temperature, for example, treatment with 2N NaOH for 1 hour and/or autoclaving. This would eliminate the danger of misfolded proteins as biohazardous waste and also help to minimize contamination that could occur when testing multiple samples. Alternatively, the substrate protein can be modified in such a way that after conversion to a misfolded form it can be easily inactivated, by for example adding a proteolytic cleavage site.

The present invention also provides a method to diagnose a disease in an animal or human by detecting the presence of a misfolded protein in a sample. These methods include, but are not limited to methods comprising one or more of steps (a), (b), (c), (d) and (e) described above. As used herein “animal” refers to any animal that is susceptible to a disease related to the aggregation of proteins, particularly misfolded protein or proteins that may sustain conformational changes that result in protein aggregation. For example, animals include but are not limited to a variety of mammals such as humans, cows, sheep, cats, pigs, deer, and elk. Detection of misfolded protein in the reaction mix is indicative of a positive diagnosis for a disease related to aggregation of proteins in the brain or other organs of the body, or it is indicative of a susceptibility to development of such disease. As defined herein “a disease related to aggregation of proteins” is any disease that is associated with protein aggregates and the protein aggregates are implicated in the onset or progression of a disease state, such diseases comprise, but are not limited to Alzheimer's disease, amyotrophic lateral sclerosis, Parkinson's disease, diabetes type 2 and the like (Soto, 2001).

It is contemplated that the detected misfolded protein could comprise abnormally folded proteins or protein fragments. For example the misfolded protein may be a wild type, variant, or mutant of mammalian amyloid β, transthyrein, immunoglobulin light chain, lysozyme, superoxide dismutase 1 (SOD1), Huntingtin protein, amylin, α-synuclein, tau, ataxin, familial British dementia protein, and the like.

It is contemplated that the method of the invention may be used to detect misfolded proteins in a wide variety of samples. In some embodiments the sample is a tissue sample from an individual. Tissues samples may comprise samples from brain, or from peripheral organs. Samples may also be obtained from biological fluids such as cerebrospinal fluid, blood, urine, milk, tears, saliva, and the like. In particular embodiments samples maybe be taken from blood. Detection of misfolded proteins in blood samples is of great interest since it can be readily taken from a living organism. Thus, the current invention could enable the detection diseases associated with aggregation of proteins from blood samples with a sensitivity sufficient to detect preclinical disease, which is an important advance in the art. In certain aspects the sample comprises blood, tears, urine, saliva, CSF, peripheral nerves, skin, muscle, or lymphoid organs, including portions thereof.

Aspects of the current invention may include disruption of protein association in the reaction mix. Disruption may be accomplished by sonication or other physical or chemical means. To prevent contamination a sonication apparatus may or may not be put in direct contact with the samples. Thus sonication with a commercially available microsonicator may be performed. The sonication apparatus may be automated and capable of programmed operation thus allowing high throughput sample amplification. For example sonication could comprise a pulse of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more seconds of sonication, or any range derivable therein, at 10%, 20%, 30%, 40%, 50%, 60%, 70%, 80%, 90%, or 100% potency, or any range derivable therein. It is also preferable that the reaction mixes be kept in a sealed environment to prevent evaporation. For example amplification may be carried out while samples are maintained in a sealed plexiglass enclosure.

In certain embodiments of the invention the parameters of the sonication step may be varied over the course of amplification. For example the sonication time and/or sonication potency maybe increased or decreased after each cycle. In certain embodiments the sonication parameters (i.e. the time and potency) could be preprogrammed for each step of cyclic amplification.

In certain aspects of the present invention it is contemplated that incubation of the reaction mixture may be at a temperature of about 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., to about 50° C., or any range derivable therein. In certain applications of the invention, the incubation is at about 37° C. It is also envisioned that the temperature may be varied throughout all or part of the process. For instance each time the reaction mix is incubated the temperature may be increased or decreased. It is also contemplated that the temperature of the reaction mix could be modified prior to disruption of the reaction mixture. In certain embodiments the temperature of the reaction mixture is monitored and/or controlled by a programmable thermostat. For example the sample may be placed in an automated thermocycler thus allowing the temperature of the reaction mixture to be programmed over the course of amplification.

It is also contemplated that incubation of the reaction mixture could be performed over a range of time periods. For example the reaction mixture may be incubated for about one minute to about 10 hours. In a certain embodiments the incubation time is about or at least about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200 minutes or hours, or any range derivable therein. In an even further embodiment, the reaction mix is incubated for about 30 minutes. It is also contemplated that the incubation time may be varied through out the amplification. For example the incubation time may be increased or decreased by an increment of time after each amplification step. In still further aspects the disruption apparatus is automated such that incubation times may be programmed.

In some embodiments of the current invention incubation and disruption (steps (b)(i) and (b)(ii)) are repeated many times, it is contemplated that they could be repeated at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 times, or any range derivable therein. It is envisioned that in some embodiments of the present invention primary amplification (step (b)) would take place over a period of about 1, 2, 3, 4, 5 days or more. In certain embodiments, the steps (c)(i) and (c)(ii), serial amplification can be repeated multiple times. For example steps (c)(i) and (c)(ii) could be repeated 1, 2, 3, 4, 5, 6, 7, 8, 9, 10, 11, 12, 13, 14, 15, 16, 17, 18, 19, 20, 21, 22, 23, 24, 25, 26, 27, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 times, or any range derivable therein. In certain aspects the additional low molecular protein is stored as lyophilized powder or tablets, and/or is kept frozen, to prevent protein degradation prior to mixing it with the reaction mix or serial reaction mix. In further embodiments the number of serial amplification steps may be preprogrammed for automated amplification.

In a further aspect of the current invention the reaction mix may further comprise a sample, a substrate protein, and a conversion buffer. In some embodiments the conversion buffer comprises a salt solution and detergents. The conversion buffer may further comprise a metal or a metal chelator. Metal chelators will reduce the active amounts of Cu²⁺, Zn²⁺ and other metals that may interfere with the amplification. In a certain embodiments the metal chelator is EDTA. The reaction mix may also comprise additional elements, for example, one or more buffers, salts, detergents, lipids, protein mixtures, nucleic acids, and/or membrane preparations.

In still further aspects, the substrate protein may be from a lysate, e.g., a cell lysate. The cell lysate may comprise a crude cell lysate or a cell lysate that has been treated in such a way as to enrich the lysate for a substrate protein. The cell lysate may be a liquid, semi-liquid, or a lyophilized protein powder or tablet. In some aspects the cell lysate comprises a brain homogenate that may or may not be subjected to purification processes. In some aspects the brain homogenate is a mammalian brain homogenate, e.g., a human brain homogenate. In still further aspects the cell lysate can be derived from the same species of organism as the test sample. The cell lysate may also be from cells that over express the substrate protein. In some embodiments the cell lysate is from cells that have been transformed with a nucleic acid expression vector that express the substrate protein. For example the substrate protein may be from cell lysate of tissue culture cells or from a tissue sample from a transgenic animal, e.g., transgenic mouse expressing a substrate protein, that over express Aβ or some other protein or protein fragment associated with protein aggregates in vivo. Also the substrate protein can be recombinantly expressed in bacteria, yeast, or insect cells. In certain aspects, the substrate protein may be synthetically produced by solid or liquid phase peptide synthesis using state-of-the-art methodology for synthesis and purification.

In yet still a further aspect of the current invention the substrate protein may comprise proteins with an amino acid sequence that is homologous to endogenous proteins. For example the substrate protein may be identical or highly similar to the endogenous proteins from mice, humans, cattle, sheep, goat, elk, or other mammals. The substrate protein may comprise Aβ with an altered amino acid sequence. For example, the substrate protein may comprise Aβ with amino acid substitutions, deletions, or insertions. Substrate proteins with alterations in the amino acid sequence may be used to study the susceptibility of certain mutant proteins for conversion to a protein or protein fragment with a propensity for aggregation or used as a more efficient substrate for replication.

In some aspects of the current invention the substrate protein may be from a cell that expresses the substrate protein as a fusion protein. For example the coding sequence for the substrate protein may be fused to other amino acid coding sequences. For example the fused amino acid coding sequences could comprise coding sequence for a reporter protein, a detectable tag, a tag for protein purification, or a localization signal. Additionally, substrate protein may be labeled for detection, i.e., detectably labeled, for example, by incorporation of radioactive amino acids or covalent modification with a fluorophore.

It is also contemplated that the substrate protein may be modified in such a way as to increase its ability to undergo conversion into a misfolded protein. In aspects the substrate protein may be pretreated to alter post-translational modifications, such as glycosylation, phosphorylation, etc. In further aspects of the current invention samples may be treated or fractionated in such a ways as to concentrate the protein of the sample prior to PMCA or saPMCA. For example protein may be concentrated by precipitation with organic solvents, immunoprecipitation, or binding to ligands shown to interact specifically with a particular protein or protein fragment associated with misfolding and/or aggregation in vivo, such as conformation specific antibodies. It is also contemplated that samples may be fractionated. For example, the fraction that is insoluble in mild detergent could be harvested.

It is contemplated that detection of amplified misfolded protein in a reaction mix or serially amplified reaction mix may be via a variety of methods that are well known to those in the art, e.g., Western blot assay, ELISA, thioflavine T binding assay, Congo red binding assay, sedimentation assay, electron microscopic assessment, spectroscopic assay, or combinations thereof. In one embodiment the reaction mix or serial reaction mix is treated with a protease, such as proteinase K, and then misfolded protein is detected by Western blot or by ELISA using anti-misfolded protein antibody. In some aspects the ELISA assay may be a two-site immunometric sandwich ELISA. In other aspects the misfolded protein may be detected by a sedimentation assay, using centrifugation to separate aggregated from soluble protein. It is also contemplated that amplified misfolded protein may be detected by methods specifically designed to detect misfolded aggregates, including binding of the amyloid aggregates to the dyes Congo red or thioflavine T and visualization of aggregates morphology by electron microscopy. Finally, amplified misfolded protein may be detected by spectroscopic methods such as atomic force microscopy, quasi-light scattering, multispectral ultraviolet fluoroscopy, confocal dual-color fluorescence correlation spectroscopy, Fourier-transformed infrared spectroscopy or capillary electrophoresis, and Fluorescence Resonance Energy Transfer (FRET) (Soto et al., 2004).

The current invention also provides an apparatus for amplification and detection of misfolded protein. The apparatus comprises a programmable microplate sonicator. The microplate sonicator may be programmed for multiple cycles, incubation times, sonication potency and sonication periods. The apparatus may further comprise an incubator capable of being programmed for a range of different incubation temperatures. In certain embodiments the apparatus may also comprise programmable robotic probes for sample and reaction mix manipulation. It is also contemplated that separation of substrate protein and misfolded protein, and detection of misfolded protein in the reaction mix may be automated. For example misfolded protein may be detected as described herein with automated ELISA methods. Wherein the substrate protein is fluorescently labeled, conformational changes may be detected by FRET and monitored “real time” as the sample is subjected to amplification.

In some aspects, the invention relates to a kit for detecting misfolded protein in a sample comprising a substrate protein. In some embodiments, the kit may further comprise: an enclosure for sample amplification such as a microtiter plate, or sample tubes; an amplification buffer that is added to the sample and substrate protein prior to amplification; positive and negative control samples for amplification, wherein the positive control sample contains misfolded protein and the negative control sample does not; a decontamination buffer for inactivation of misfolded protein, for example an spray, solution, or wipe comprising 2N sodium hydroxide; materials for separating misfolded protein from substrate, for instance a proteinase K digestion buffer, or a misfolded protein fractionation buffer; materials for detection misfolded protein, for example conformation specific antibodies for Western blotting or ELISA tests, or reagents for Congo red or thioflavine T binding assays.

As used herein, “sensitivity” refers to the ability of an assay to detect the presence of a misfolded protein or protein fragment (i.e., to give a high percentage of true positive reactions and a low percentage of false negative reactions). As used herein, specificity refers to the ability of an assay to reliably distinguish between misfolded protein and properly folded protein (i.e., to give a low percentage of false positive reactions and a high percentage of true negative reactions). Aspects of the invention include methods capable of detecting less than 2, 5, 10, 50, 100, 200, 500 attograms (ag), 1, 0.9, 0.8, 0.7, 0.6, 0.5, femtogram (fg) or less of misfolded protein in a 10 μl sample. In further aspects, the methods are capable of detecting at least about 10, 50, 100, or 1000 or more molecules of misfolded protein or less in a sample (e.g., per 20 μl of sample). In still further aspects, the methods of the invention are capable of detecting misfolded protein in sample dilutions of 1×10⁻⁷, 5×10⁻⁷, 1×10⁻⁸, 5×10⁻⁸, 1×10⁻⁹ 5×10⁻⁹, 1×10⁻¹⁰, 5×10⁻¹⁰, 1×10⁻¹¹, 5×10⁻¹¹, 1×10⁻², 5×10⁻¹², or more of sample (e.g., blood or brain tissue), including all values in between. Methods of the invention will typically be capable of a 4×10⁵, 1×10⁶, 5×10⁶, 1×10⁷, 1×10⁸, 1×10⁹, 3×10⁹ or greater fold increase, including all values in between, in sensitivity as compared to standard methodologies, such as ELISA. Embodiments of the invention include a specificity of detection greater than 90%, 92%, 95%, 98%, 99% up to 100% of assays capable of distinguishing misfolded and properly folded protein.

Embodiments discussed in the context of methods and/or composition of the invention may be employed with respect to any other method or composition described in this application. Thus, an embodiment pertaining to one method or composition may be applied to other methods and compositions of the invention as well.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Illustration of the seeding-nucleation model for protein aggregation.

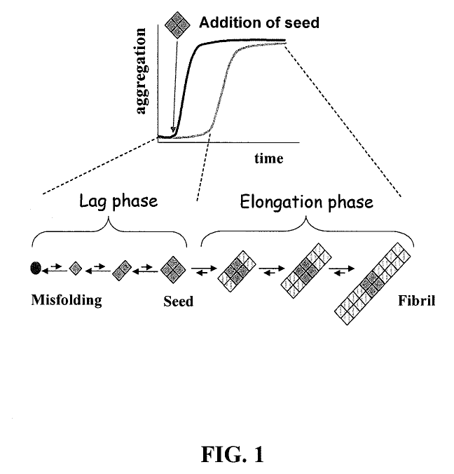

FIG. 2. Schematic representation of one methodology employed to prepare the substrate seed free (SF) Aβ.

FIG. 3. Comparison of the sensitivity of detection of various methods for detecting prions.

FIG. 4. Seeded aggregation of low concentrations of Aβ. Aβ42 (500 nM) was incubated for various times in the absence or the presence of preformed seeds (as indicated in the legend on the right side) at 37° C. Thereafter the quantity of peptide remaining soluble was determined by sedimentation assays.

### DETAILED DESCRIPTION OF THE INVENTION

Neurodegenerative diseases, including Alzheimer's, Parkinson's, Huntington's, ALS, and TSE, are associated with protein misfolding events leading to the formation of amyloid fibrils and other pathologic protein aggregates (Soto, 2003). The gross histological signs of abnormal protein folding and assembly are unmistakable-senile plaques, neurofibrillary tangles, Lewy bodies, intracellular inclusions, and spongiform degeneration. Depending on the protein and the tissues affected, abnormal folding can cause injury and death, both at the cellular and organism level.

Disclosed herein is a method to detect misfolded protein in a sample; this method can be used to diagnose a variety of diseases in animals or humans or to indicate a propensity for development of disease at a later date, e.g., Alzheimer's disease (AD). The methods for detection of misfolded protein of the invention improve sensitivity and reduce the time necessary for high sensitivity detection of misfolded protein in samples. The current invention enables high throughput, accurate, and sensitive screening of samples, as well as diagnosis of clinical disease or a propensity for developing such, particularly in asymptomatic subjects.

It is also contemplated that the diagnostic methods described could be applied to humans and human diseases. Misfolded protein diseases that could be diagnosed in humans comprising Parkinson's, Huntington's, diabetes type 2, ALS, Alzheimer's, light chain amyloidosis, secondary systemic amyloidosis, dyalisis-related amyloidosis and other diseases known to be associated with protein aggregation (Soto, 2001). Again the method of the invention offers significant advantages over currently available methods for diagnosis of these disorders. The invention offers an objective method by which positive diagnosis may be made with a reduced chance of false positive or negative results. Additionally the sensitivity of the test enables the detection of disease from peripheral tissues, such as blood, which is much less invasive and expensive than current brain biopsy or imaging procedures. The invention also provides sensitivity that is high enough such that disease may be detected and diagnosed long before the onset of clinical symptoms.

Misfolded proteins such as misfolded Aβ, also known as beta-amyloid, are known to be associated with Alzheimer's disease and may be detected using the methods described herein. This method could further be used as a diagnostic test for Alzheimer's disease. Diagnosis of Alzheimer's is currently based primarily on cognitive tests, and a biochemical testing procedure would be a great advantage.

Another application of the present invention is as a high throughput method of screening for compounds that enhance or inhibit conversion of substrate protein into misfolded proteins. In this respect it is envisioned that the reaction mixture could further comprise a test compound. Control reaction mixtures and reaction mixtures including the test compound could be assessed for levels of misfolded protein following amplification. Wherein a difference between the levels of misfolded protein in the test versus control reaction mixtures is detected, compounds could be identified that either enhance or inhibit conversion of substrate protein to a misfolded state. In further aspects, samples from control and test reaction mixtures may be taken after two, three, four or more amplification steps to determine a rate of misfolded protein replication. By comparing the rate of control misfolded protein replication versus the rate of propagation in the presence of a test compound candidate modifiers could be quantitatively assessed for their effect on misfolded protein replication.

Diagnosis of Alzheimer's disease is most often made in the moderate stage. Typically the symptoms of AD are cognitive dysfunction or deficiency, and include dementia confirmed by medical and psychological exams, problems in at least two areas of mental functioning, progressive loss of memory and other mental functions, symptoms that began between the ages of 40 and 90, no other disorders that might account for the dementia, and no other conditions that may mimic dementia including hypothyroidism, overmedication, drug-drug interactions, vitamin B12 deficiency, or depression. The moderate stage of AD is often recognized when sufferers or family and friends begin to recognize cognitive impairment or symptoms, and will consult their doctor. To diagnose Alzheimer's disease, doctors use a series of tests and tools that evaluate physical, behavioral, and emotional response.

Common diagnostic tests administered in the doctor's office may include: (1) Mini-Mental State Examination (MMSE)—MMSE consists of 11 questions that cover five cognitive areas: orientation, registration (ability to recognize and name specific items), attention, recall, and language. It's relatively easy for doctors to administer, and takes only 5 to 10 minutes. This test is used for diagnosis in the mild, moderate and severe stages of Alzheimer's disease. (2) The Clock Test—This is an easy-to-administer indicator of cognitive decline where patients are asked to draw a clock, including all the numbers and a specific time. Patients are then scored on numbers included, location of the numbers, and location and size of clock hands. This test is used to assess patients with mild, moderate or severe Alzheimer's disease. (3) Functional Assessment Staging (FAST)—Rather than diagnosis, FAST is used for determining which stage a patient diagnosed with Alzheimer's disease is in. This scale assesses a range of activities, including dressing, continence, and ability to speak, sit up, and smile. This test is used to assess patients with mild, moderate or severe Alzheimer's disease. Other tests may be used such as: (4) Alzheimer's Disease Assessment Scale, Cognitive Subscale (ADAS-Cog)—ADAS-Cog is a highly accurate scale in diagnosing and staging mild to moderate Alzheimer's disease. It's used to gauge change in cognition, with a focus on memory and language. One of the limitations of this scale is that there is a “floor effect”, which means that when a patient reaches a certain point, the scale can no longer measure cognitive decline. (5) Severe Impairment Battery (SIB)—SIB was designed to assess cognitive functioning in patients who are too impaired to take other standardized cognitive scales. It consists of 40 questions (some with multiple parts), which measure patients' cognitive range in areas such as orientation, language, memory, and attention. This test is used to assess patients in the moderate to severe stages of Alzheimer's disease. (6) Modified Alzheimer's Disease Cooperative Study—Activities of Daily Living Inventory (ADCS-ADL)—ADCS-ADL measures a patient's functional capacity over a broad range of dementia severities. Patients are evaluated on a series of questions designed to determine their ability to perform specific activities of daily living, activities which include bathing, dressing, eating, walking, and more. This test is used to assess patients in the moderate to severe stages of Alzheimer's disease. (7) Behavioral Rating Scale for Geriatric Patients (BGP)—BGP assesses both functional and behavioral disturbances in geriatric patients. Assessments include physical disabilities, abilities to perform activities of daily living (ADLs), and level of activity vs. inactivity. This test is used to assess patients with severe Alzheimer's disease. (8) Neuropsychiatric Inventory (NPI)—NPI evaluates behavioral disturbances with a 12-item questionnaire. The items include delusions or paranoia, hallucinations, agitation or aggression, depressed mood, anxiety, elation or euphoria, apathy or indifference, disinhibition, irritability, motor disturbance, nighttime behaviors, and appetite problems. This test is used to assess patients with mild, moderate or severe Alzheimer's disease. (9) Clinicians' Interview-Based Impression of Change Plus Caregiver Input (CIBIC-Plus)—CIBIC-Plus measures the overall improvement or decline of a patient's cognitive function through a series of interview questions. Through this test, both the patient and caregiver are interviewed. This test is used to assess patients with mild, moderate or severe Alzheimer's disease.

In addition a medical and family health history; a routine physical exam; a test of physical sensation; sense of balance, and other functions controlled by the central nervous system; a brain scan to rule out other causes of dementia, such as stroke; a psychiatric evaluation, to assess mood and other emotional factors that may lead to a positive diagnosis; and interviews with family members and friends that provide insight into behavioral changes, if the patient or family agrees.

### I. Protein Sources

A. Sources of Substrate Protein

As detailed above, a variety of sources may be used to obtain substrate protein for use in the methods of the invention.

For instance the protein maybe endogenously expressed in cells and these cells used to make a lysate that provides the substrate protein. The lysate may be from tissue culture cells, or extracted from whole organisms, organs, or tissues. For example, in the case where the substrate protein is Aβ, brain homogenates may be used. These brain homogenates may be mammalian brain homogenates, and in certain aspects the homogenates are from the same species as the particular sample being tested or from transgenic mice engineered to express Aβ from the specie to be tested. It is envisioned that in addition to using crude cell lysates partially purified protein may also be used, as well as synthetic peptides.

In some embodiments of the invention the source of the substrate protein maybe from cells made or engineered to over express a protein. For instance cells may be transformed with a nucleic acid vector that expresses the substrate protein, for example Aβ. These cells may comprise mammalian cells, bacterial cells, yeast cell, insect cells, whole organisms (such as transgenic mice), or other cells that may be a useful source of the substrate protein. Raw cell lysates or purified substrate protein from expressing cells may be used as the source of the substrate protein.

In some embodiments of the invention the source of the substrate protein maybe a synthetic peptide produced by state-of-the-art liquid-phase or solid-phase techniques frequently used to synthesize peptides or short proteins. The synthetic peptides are then purified by reverse-phase HPLC.

As indicated above it may in some cases the substrate protein may be further processed, e.g., deglycosylated or treated with another enzyme or chemical. For example substrate protein may be treated with peptide N-glycosidase F (New England Biolabs, Beverly, Mass.) according to the manufacturers instructions. For example, incubation of Aβ for about 2 h at 37° C. results in significant deglycosylation.

Generally, “purified” will refer to a substrate protein composition that has been subjected to fractionation or isolation to remove various other protein or peptide components, and which composition substantially retains substrate protein, as may be assessed, for example, by Western blot to detect the substrate protein.

To purify substrate protein from natural or recombinant composition the composition will be subjected to fractionation to remove various other components from the composition. Various techniques suitable for use in protein purification will be well known to those of skill in the art. These include, for example, precipitation with ammonium sulfate, PTA, PEG, antibodies, and the like, or by heat denaturation followed by centrifugation; chromatography steps such as ion exchange, gel filtration, size exclusion, reverse phase, hydroxylapatite, lectin affinity and other affinity chromatography steps; isoelectric focusing; gel electrophoresis; and combinations of such and other techniques.

In some cases it may be preferable that the recombinant protein be fused with additional amino acid sequence. For example over expressed protein may be tagged for purification or to facilitate detection of the protein in a sample. Some possible fusion proteins that could be generated include histidine tags, Glutathione S-transferase (GST), Maltose binding protein (MBP)), green fluorescent protein (GFP), Flag, and myc tagged proteins, to name a few. These additional sequences may be used to aid in purification and/or detection of the recombinant protein, and in some cases may then be removed by protease cleavage. For example coding sequence for a specific protease cleavage site may be inserted between the substrate protein coding sequence and the purification tag coding sequence. One example for such a sequence is the cleavage site for thrombin. Thus fusion proteins may be cleaved with the protease to free the substrate protein from the purification tag.

After the substrate protein is produced and purified, an essential part to enable efficient amplification is to treat the material by a procedure to remove the protein aggregates non-specifically formed during production and isolation of the protein to obtain a substrate preparation termed seed-free (SF) Aβ fraction. Seed free typically refers to a substrate solution containing less than about 0%, 0.01%, 0.1%, 0.5%, 1% of detectable aggregates. This is important because otherwise, the non-specific aggregates may mask the effect of misfolded oligomers present in the sample. For this purpose, the Aβ preparation is incubated with a solvent that promotes disassembly of preformed aggregates, such as 10 mM NaOH, pH 12. Other solvents to use include different concentrations of sodium hydroxide, hexafluoroisopropanol, trifluoroacetic acid, acetonitrile, dimethylsulfoxide, guanidine hydrochloride, urea, formic acid, hydrochloride acid, ammonium hydroxide, trifluoroethanol, etc. After dissolution, the samples are subjected to size exclusion chromatography or filtration through 10 kDa cut off filters. This preparation results in the SF Aβ fraction, which is kept lyophilized to avoid re-aggregation.

When a substrate protein is highly purified the reaction mix may further comprise additional cell lysate to provide secondary factors important for conversion. For example, brain homogenate from an unaffected animal may be used to supplement the reaction mix. It is contemplated that the method of the invention is used to identify co-factors important in pathogenic conversion of various proteins.

Any of the wide variety of vectors known to those of skill in the art may be used to over express substrate protein. For example, plasmids or viral vectors may be used. It is well understood to those of skill in the art that these vectors may be introduced into cells by a variety of methods including, but not limited to, transfection (e.g., by liposome, calcium phosphate, electroporation, particle bombardment, etc.), transformation, and viral transduction.

Substrate protein may further comprise proteins that have amino acid sequence containing substitutions, insertions, deletions, and stop codons, as compared to a wild type or non-pathogenic sequence. In certain embodiments of the invention, a protease cleavage sequence may be added to allow inactivation of a protein after it is converted into a misfolded protein. A non-limiting example of such cleavage sequences include those recognized by Thrombin, Tobacco Etch Virus (Life Technologies, Gaithersburg, Md.) or Factor Xa (New England Biolabs, Beverley, Mass.) proteases may be inserted into the sequence.

In certain embodiments changes may be made in the substrate protein coding sequence. For example mutations could be made to match a variety of mutations and polymorphisms known for various mammalian genes. It is contemplated that cells or animals expressing these altered genes may be used as a source of the substrate protein. Cells may endogenously express the mutant protein gene or be manipulated to express a mutant protein by the introduction of an expression vector. Use of a mutated substrate protein may be of particular advantage, as it is possible that these proteins may be more easily converted to a misfolded protein, and thus may further enhance the sensitivity of the methods of the invention.

It is contemplated that the method of the current invention may be used to test the effect of mutations on the conversion rates of substrate proteins to protein aggregates. For example, a mutant substrate protein and wild type substrate protein can be mixed with equal amounts of misfolded protein and amplification performed. By comparing the rate of misfolded protein replication in samples with mutant substrate protein versus wild type substrate protein mutations could be identified that modulate the ability of misfolded protein to replicate.

B. Sources of Samples for Amplification Assay

As described above it is contemplated that samples used in the methods of the invention may essentially comprise any composition capable of being contaminated with a misfolded protein. Such compositions could comprise tissue samples including, but not limited to, blood, lymph node, brain, spinal cord, tonsil, spleen, skin, muscle, appendix, olfactory epithelium, cerebrospinal fluid, urine, milk, intestine, tears and/or saliva samples; food; and environmental samples.

### II. Detecting Misfolded Proteins

Direct and indirect methods may be used for detection of misfolded protein in a sample, a reaction mix, or a serial reaction mix. For methods in which a misfolded protein is directly detected, separation of newly formed misfolded protein from remaining substrate protein may be performed. This is typically accomplished based on the different nature of misfolded protein versus substrate protein, for instance misfolded protein may be highly insoluble and resistant to protease treatment. Therefore, separation may be by protease treatment, size based chromatography, differential centrifugation in a detergent, or other known methods specifically designed to identify the abnormal folding of the protein, including combinations of these techniques.

In the case where misfolded protein and substrate protein are separated by protease treatment, reaction mixtures are incubated with, for example, Proteinase K (PK). An exemplary proteinase treatment comprises digestion of the protein in the reaction mixture with 1-100 μg/ml of proteinase K (PK) for about 1 hour at 45° C. Reactions with PK may be stopped prior to assessment of misfolded protein levels by addition of PMSF or electrophoresis sample buffer. Incubation at 45° C. with 1-100 μg/ml of PK is sufficient to remove substrate protein, but does not degrade the misfolded aggregated protein.

In some cases substrate protein may be separated from misfolded protein by fractionation. Differential solubility may also be used. An exemplary procedure comprises centrifuging the reaction mixture at 100,000×g for 1 hr in a Biosafe Optima MAX ultracentrifuge (Beckman Coulter, Fullerton, Calif.) and the pellet, which contains the misfolded protein, is resuspended and analyzed for misfolded protein.

Misfolded protein might also be separated from the substrate protein by the use of ligands that specifically bind and precipitate the misfolded form of the protein, including conformational antibodies, certain nucleic acids, plasminogen, organic solvents and/or various peptide fragments (Soto et al., 2004).

A. Western Blotting

Reaction mixtures fractioned or treated with protease to remove non-aggregated proteins may be subjected to Western blot for detection of misfolded protein. Typical Western blot procedures begin with fractionating proteins by sodium dodecyl sulphate-polyacrylamide gel electrophoresis (SDS-PAGE) under reducing conditions. The proteins are then electroblotted onto a membrane, such as nitrocellulose or PVDF and probed, under conditions effective to allow immune complex (antigen/antibody) formation, with an anti-misfolded protein antibody. Following complex formation the membrane is washed to remove non-complexed material. A preferred washing procedure includes washing with a solution such as PBS/Tween, or borate buffer. The immunoreactive bands are visualized by a variety of assays known to those in the art. For example the enhanced chemiluminescence assay (ECL) (Amersham, Piscataway, N.J.).

Misfolded protein concentration may be estimated by Western blot followed by densitometric analysis, and comparison to Western blots of samples for which the concentration of misfolded protein is known. For example this may be accomplished by scanning data into a computer followed by analysis with quantitation software. To obtain a reliable and robust quantification, several different dilutions of the sample are typically analyzed in the same gel.

B. ELISA

As detailed above, immunoassays in their most simple and direct sense are binding assays. Certain preferred immunoassays are the various types of enzyme linked immunosorbent assays (ELISAs) and radioimmunoassays (RIA).

In one exemplary ELISA, the anti-substrate protein antibodies are immobilized onto a selected surface exhibiting protein affinity, such as a well in a polystyrene microtiter plate. Then, reaction mixture after amplification is added to the wells. After binding and washing to remove non-specifically bound immune complexes, the bound misfolded protein may be detected. Detection is generally achieved by the addition of another protein antibody that is linked to a detectable label. This type of ELISA is a simple “sandwich ELISA.” Detection may also be achieved by the addition of a second anti-substrate protein antibody, followed by the addition of a third antibody that has binding affinity for the second antibody, with the third antibody being linked to a detectable label.

In another exemplary ELISA, the reaction mixture after amplification is immobilized onto the well surface and then contacted with the anti-substrate protein antibodies. After binding and washing to remove non-specifically bound immune complexes, the bound anti-misfolded protein antibodies are detected. Where the initial anti-misfolded protein antibodies are linked to a detectable label, the immune complexes may be detected directly. Again, the immune complexes may be detected using a second antibody that has binding affinity for the first anti-substrate protein antibody, with the second antibody being linked to a detectable label.

Another ELISA in which protein of the reaction mix is immobilized, involves the use of antibody competition in the detection. In this ELISA, labeled antibodies against misfolded protein are added to the wells, allowed to bind, and detected by means of their label. The amount of misfolded protein antigen in a given reaction mix is then determined by mixing it with the labeled antibodies against misfolded protein before or during incubation with coated wells. The presence of misfolded protein in the sample acts to reduce the amount of antibody against misfolded protein available for binding to the well and thus reduces the ultimate signal. Thus the amount of misfolded protein in the sample may be quantified.

Irrespective of the format employed, ELISAs have certain features in common, such as coating, incubating or binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, or a secondary binding ligand or antibody in conjunction with a labeled tertiary antibody or third binding ligand.

“Under conditions effective to allow immune complex (antigen/antibody) formation” means that the conditions preferably include diluting the antigens and antibodies with solutions such as BSA, bovine gamma globulin (BGG) and phosphate buffered saline (PBS)/Tween. These added agents also tend to assist in the reduction of nonspecific background.

The “suitable” conditions also mean that the incubation is at a temperature and for a period of time sufficient to allow effective binding. Incubation steps are typically from about 1 to 2 to 4 hours, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact and incubate the first or second immune complex with a urease, glucose oxidase, alkaline phosphatase, or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azino-di-(3-ethyl-benzthiazoline-6-sulfonic acid [ABTS] and H₂O₂, in the case of peroxidase as the enzyme label. Quantification is then achieved by measuring the degree of color generation, e.g., using a visible spectra spectrophotometer.

C. Amyloid Detection Assays

The formation of misfolded aggregates can be also quantitated by methods specifically designed to measure amyloid-like aggregates. With this purpose, four different assays can be used: (a) A fluorometric assay based in the fluorescence emission by thioflavine T, following a protocol modified from previous publications (Soto et al., 1995a; Soto et al., 1998) and optimized for medium throughput using 96-wells ELISA plates. Thioflavine T binds specifically to amyloid and this binding produces a shift in its emission spectrum and a fluorescent enhancement proportional to the amount of amyloid formed (Naiki et al., 1989; LeVine, 1993). (b) A spectrophotometric assay based on the specific interaction of Congo red with amyloid fibrils. After the incubation period, Congo red (e.g., 2 μl of 1.5 mg/ml) is added to each sample and incubated in the dark, e.g., for 1 h. Thereafter samples are centrifuged at 15,000 rpm for 10 min and the absorbance of the supernatant is measured at 490 nm. The amount of amyloid formed is directly proportional to the decrease in the supernatant absorbance (Klunk et al., 1999). (c) A sedimentation assay as described (Soto et al., 1995b) can also be used. Briefly, after incubation samples are centrifuged at 15,000 rpm for 10 min to separate the soluble and aggregated peptide. The amount of material remaining soluble will be quantitated by ELISA or reverse phase HPLC. (d) Electron microscopic examination after negative staining, using standard protocols may also be used (Soto et al., 1995a; Soto et al., 1998). Briefly, the incubated samples are placed onto carbon formar-coated 300-mesh nickel grids and stained, e.g., for 60 seconds with 2% uranyl acetate under a vapor of 2% glutaraldehyde. Grids are visualized on a Zeiss EM 10 electron microscope at 80 kV or similar device.

D. Protein Labeling

In certain aspects of the present invention, the substrate protein can be labeled to enable high sensitivity of detection of protein that is converted into misfolded protein or protein aggregates. For example, substrate protein may be radioactively labeled, epitope tagged, or fluorescently labeled. The label may be detected directly or indirectly. Radioactive labels include, but are not limited to ¹²⁵I, ³²P, ³H, ¹⁴C and ³⁵S.

The mixture containing the labeled protein is subjected to amplification and the product detected with high sensitivity by following conversion of the labeled protein after removal of the unconverted protein, for example by proteolysis. Alternatively, the protein could be labeled in such a way that a signal can be detected upon the conformational changes induced during conversion. An example of this is the use of FRET technology, in which the protein is labeled by two appropriate fluorophors, which upon refolding become close enough to exchange fluorescence energy (see for example U.S. Pat. No. 6,855,503).

One class of dyes that have been developed to give large and different Stokes shifts, based on the Fluorescence Resonance Energy Transfer (FRET) mechanism and used in the simultaneous detection of differently labeled samples in a mixture, are the ET (Energy Transfer) dyes. These ET dyes include a complex molecular structure consisting of a donor fluorophore and an acceptor fluorophore as well as a labeling function to allow their conjugation to biomolecules of interests. Upon excitation of the donor fluorophore, the energy absorbed by the donor is transferred by the FRET mechanism to the acceptor fluorophore and causes it to fluoresce. Different acceptors can be used with a single donor to form a set of ET dyes so that when the set is excited at one single donor frequency, various emissions can be observed depending on the choice of the acceptors. Upon quantification of these different emissions, changes in the folding of a labeled protein may be rapidly determined. Some exemplary dyes that may be used comprise BODIPY FL, fluorescein, tetmethylrhodamine, IAEDANS, EDANS or DABCYL. Other dyes have also been used for FRET for examples dyes disclosed in U.S. Pat. Nos. 5,688,648, 6,150,107, 6,008,373 and 5,863,727 and in PCT publications WO 00/13026, and WO 01/19841, all incorporated herein by reference.

### III. Antibody Generation

In certain embodiments, the present invention involves antibodies. For example, antibodies are used in many of the method for detecting misfolded protein (e.g. Western blot and ELISA). In addition to antibodies generated against full length proteins, antibodies also may be generated in response to smaller constructs comprising epitopic core regions, including wild-type and mutant epitopes.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

Monoclonal antibodies (mAbs) are recognized to have certain advantages, e.g., reproducibility and large-scale production, and their use is generally preferred. The invention thus provides monoclonal antibodies of the human, murine, monkey, rat, hamster, rabbit and even chicken origin.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Harlow and Lane, 1988; incorporated herein by reference).

The methods for generating monoclonal antibodies (mAbs) generally begin along the same lines as those for preparing polyclonal antibodies. Briefly, a polyclonal antibody may be prepared by immunizing an animal with an immunogenic polypeptide composition in accordance with the present invention and collecting antisera from that immunized animal. Alternatively, in some embodiments of the present invention, serum is collected from persons who may have been exposed to a particular antigen. Persons exposed to a particular antigen may have developed polyclonal antibodies to a peptide, polypeptide, or protein. In some embodiments of the invention polyclonal serum from such an exposed person(s) is used to identify antigenic regions in a misfolded protein through the use of immunodetection methods.

A wide range of animal species can be used for the production of antisera. Typically the animal used for production of antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin also can be used as carriers. Means for conjugating a polypeptide to a carrier protein are well known in the art and include glutaraldehyde, m-maleimidobenzoyl-N-hydroxysuccinimide ester, carbodiimide and bis-biazotized benzidine.

As also well known in the art, the immunogenicity of a particular immunogen composition can be enhanced by the use of non-specific stimulators of the immune response, known as adjuvants. Suitable molecular adjuvants include all acceptable immunostimulatory compounds, such as cytokines, toxins or synthetic compositions.

Adjuvants that may be used include IL-1, IL-2, IL-4, IL-7, IL-12, γ-interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion also is contemplated. MHC antigens may even be used. Exemplary, often preferred adjuvants include complete Freund's adjuvant (a non-specific stimulator of the immune response containing killed Mycobacterium tuberculosis), incomplete Freund's adjuvants and aluminum hydroxide adjuvant.

In addition to adjuvants, it may be desirable to co-administer biologic response modifiers (BRM), which have been shown to upregulate T cell immunity or down-regulate suppressor cell activity. Such BRMs include, but are not limited to, Cimetidine (CIM; 1200 mg/d) (Smith/Kline, Pa.); low-dose Cyclophosphamide (CYP; 300 mg/m²) (Johnson/Mead, N.J.), cytokines such as γ-interferon, IL-2, or IL-12 or genes encoding proteins involved in immune helper functions, such as B-7.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection also may be given. The process of boosting and titering is repeated until a suitable titer is achieved. When a desired level of immunogenicity is obtained, the immunized animal can be bled and the serum isolated and stored, and/or the animal can be used to generate mAbs.

mAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified polypeptide, peptide or domain, be it a wild-type or mutant composition. The immunizing composition is administered in a manner effective to stimulate antibody producing cells.

mAbs may be further purified, if desired, using filtration, centrifugation and various chromatographic methods such as HPLC or affinity chromatography. Fragments of the monoclonal antibodies of the invention can be obtained from the monoclonal antibodies so produced by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It also is contemplated that a molecular cloning approach may be used to generate mAbs. For this, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 10⁴ times as many antibodies can be produced and screened in a single round, and that new specificities are generated by H and L chain combination which further increases the chance of finding appropriate antibodies.

### IV. Screening for Modulators of Protein Misfolding

As described above the current invention may be used to identify compounds that modify the ability of misfolded proteins to replicate, such compounds would be candidates for treatment of misfolded protein or protein aggregate mediated disease. It is envisioned that the method for screening compounds could comprise performing amplification on control reaction mixtures and reaction mixtures including the test compound could be accessed for levels of misfolded protein following amplification. Wherein a difference between the levels of misfolded protein in the test versus control reaction mixtures is detected, compounds could be identified that either enhance or inhibit conversion of substrate protein to misfolded protein. These assays may comprise random screening of large libraries of candidate substances; alternatively, the assays may be used to focus on particular classes of compounds selected with an eye towards structural attributes that are believed to make them more likely to modulate the function of misfolded proteins.

By function, it is meant that one may determine the efficiency of conversion by assaying conversion of a standard amount of substrate protein into misfolded protein by a known amount of misfolded protein. This may be determined by, for instance, quantitating the amount of misfolded protein in a reaction mix following a certain number of cycles of amplification. Due to the rapid, high throughput nature of amplification assays it is envisioned that panels of potential misfolded protein replication modulators may be screened.

It will, of course, be understood that all the screening methods of the present invention are useful in themselves notwithstanding the fact that effective candidates may not be found or identified. The invention provides methods for screening for such candidates, not solely methods of finding them.

As used herein the term “candidate substance” refers to any molecule that may potentially inhibit or enhance misfolded protein function activity. The candidate substance may be a protein or fragment thereof, a small molecule, or even a nucleic acid molecule. Using lead compounds to help develop improved compounds is know as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It is also possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled on active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources (including leaves and bark), and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compound(s) that may be designed through rational drug design starting from known inhibitors or stimulators. Other suitable modulators include antibodies (including single chain antibodies), each of which would be specific for the target molecule. Such compounds are described in greater detail elsewhere in this document.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators. Preferred modulators of misfolded protein replication would have the ability to cross the blood-brain barrier since a large number of misfolded protein manifest themselves in the central nervous system.

An inhibitor according to the present invention may be one which exerts its activity directly on the misfolded protein, on the substrate protein or on factors required for the conversion of substrate protein to misfolded protein. Regardless of the type of inhibitor or activator identified by the present screening methods, the effect of the inhibition or activation by such a compound results in altered misfolded protein amplification or replication as compared to that observed in the absence of the added candidate substance.

### V. Kits

Any of the compositions described herein may be comprised in a kit. In a non-limiting example, substrate protein, misfolded protein conversion factors, decontamination solution, and/or conversion buffer with or without a metal chelator are provided in a kit. The kit may further comprise reagents for expressing or purifying substrate protein. The kit may also comprise reagents that may be used to label the substrate protein, with for example, radioisotopes or fluorophors. The kit may also include reagents to detect the misfolded protein.

Kits for implementing methods of the invention described herein are specifically contemplated. In some embodiments, there are kits for amplification and detection of misfolded protein in a sample. In these embodiments, a kit can comprise, in suitable container means, 1, 2, 3, 4, 5, 6, 7, 8, 9, 10, 11, 12 or more of the following: 1) a conversion buffer; 2) substrate protein; 3) decontamination solution; 4) a positive control containing a misfolded protein; 5) a negative control not containing a misfolded protein; or 6) reagents for detection of misfolded protein.

Reagents for the detection of misfolded protein can comprise one or more of the following: pre-coated microtiter plates for ELISA; antibodies for use in ELISA, or Western blot detection methods; thioflavine T, Congo red, or reagents for electron microscopy, etc.

Additionally, kits of the invention may contain one or more of the following: protease free water; copper salts for inhibiting misfolded protein replication; EDTA solutions for enhancing misfolded protein replication; Proteinase K for the separation of misfolded protein from substrate protein; fractionation buffers for the separation of misfolded protein from substrate, modified, or labeled proteins (increase sensitivity of detection); or conversion factors (enhance efficiency of amplification).

In certain embodiments the conversion buffer may be supplied in a “ready for amplification format” where it is allocated in a microtiter plate such that the sample and substrate protein may be added to a first well, and subjected to primary amplification. There after a portion of the reaction mix is moved to an adjacent well and additional substrate protein added for further amplification if needed. These steps may be repeated across the microtiter plate for multiple serial amplifications.

The components of the kits may be packaged either in aqueous media or in lyophilized form. The container means of the kits will generally include at least one vial, test tube, plate, flask, bottle, syringe or other container means, into which a component may be placed, and preferably, suitably aliquoted. Where there is more than one component in the kit (labeling reagent and label may be packaged together), the kit also will generally contain a second, third or other additional containers into which the additional components may be separately placed. However, various combinations of components may be comprised in a vial. The kits of the present invention also will typically include a means for containing proteins, and any other reagent containers in close confinement for commercial sale. Such containers may include injection or blow-molded plastic containers into which the desired containers are retained.

When components of the kit are provided in one and/or more liquid solutions, the liquid solution is typically an aqueous solution that is proteinase free and may be sterile. In some cases proteinaceous compositions may be lyophilized to prevent degradation and/or the kit or components thereof may be stored at a low temperature (i.e. less than about 4° C.). When reagents and/or components are provided as a dry powder and/or tablets, the powder can be reconstituted by the addition of a suitable solvent. It is envisioned that the solvent may also be provided in another container means.

### EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Material and Methods for Detecting Aβ

Biological samples. Several postmortem frozen brain samples from normal individuals and patients affected by AD, Creutzfeldt-Jakob disease (CJD), Parkinson, Huntington disease, and amyotrophic lateral sclerosis are available to the inventors. Cerebrospinal fluid (CSF) and blood samples can be obtained from hospital patients (Alzheimer's clinic, neurology clinic and geriatric clinic), following the proper procedures to warranty permission, confidentiality, and anonymity. The inventors have available colonies of single transgenic mice expressing the Swedish mutation in the human amyloid precursor protein gene (Tg2576) and double transgenic mice expressing the Swedish APP mutation in conjunction with the A246E mutation in the human presenilin 1 gene.

Synthetic peptides. Aβ1-40 and Aβ1-42 can be synthesized using solid phase N-tert-butyloxycarbonyl chemistry. Peptide purification is carried out by reverse-phase HPLC. The final products are lyophilized and characterized by amino acid analysis and laser desorption mass spectrometry.

Aβ amplification assay. Low concentrations (50-500 nM) of SF Aβ dissolved in 0.1 M sodium phosphate, pH 7.5 are incubated alone or with samples putatively containing seeds of misfolded protein. The final volume is 100 μl and samples are incubated with or without shaking, and with or without repetitive sonication pulses. Samples are loaded onto non-adherent ELISA plate wells. Plates are placed on the plate holder of a microsonicator Misonix and programmed to perform cycles of 1 hour incubation at 37° C. followed by a pulse of sonication. At the end of the amplification procedure the samples are centrifuged at 15,000 rpm and the quantity of peptide remaining in solution measured by an ultra-sensitive sandwich ELISA assay.

Detection of Aβ by sandwich ELISA. Aβ remaining in solution after amplification can be measured by an ultra-sensitive sandwich ELISA assay purchased from SIGMA, following the manufacturer specifications. Briefly, the supernatant of the samples after amplification is loaded onto ELISA plates precoated with a monoclonal antibody specific for NH₂ terminus of human Aβ. After a 3 hour incubation at room temperature, plates are washed 4 times and incubated with a detection antibody, produced in rabbit, which recognizes specifically human Aβ42. Samples are incubated for 1 hour at room temperature and after washing, the secondary anti-rabbit IgG conjugated with horseradish peroxidase is added. After 30 min incubation, followed by 4 washes, the plates are incubated with stabilized chromogen (substrate) for 30 min. The reaction is stopped with stop solution and color read at 450 nm. This assay is typically able to detect, reproducibly, as little as 1 ng of Aβ.

Characterization of Aβ aggregates. In some experiments, the amyloid nature of the aggregates was evaluated by three alternative protocols: (A) a fluorometric assay based in the fluorescence emission by thioflavine T, as previously described in Soto et al. (1995). (B) A spectrophotometric assay based on the specific binding of Congo red with amyloid fibrils, using the formula Cb (μM)=(A₅₄₁/47,800)−(A₄₀₃/68,300)−(A₄₀₃/86,200), as reported in Klunk et al (1999). (C) Electron microscopy after negative staining as described in Soto et al. (1995).

Preparation of synthetic Aβ seeds. Solutions of Aβ1-42 (1 mg/ml) were incubated during 5 days at 37° C. in 0.1 M sodium phosphate, pH 7.5. Thereafter, samples were centrifuged at 15,000 rpm for 10 min to separate the soluble and aggregated peptide (i.e., seeds). The pellet was resuspended in buffer and subjected to a 2 min sonication to cut down large fibrils into smaller polymers. The efficiency of the procedure can be evaluated by electron microscopy.

Isolation of Aβ oligomers and protofibrils. Protofibrils and soluble oligomers can be prepared and purified as described in Walsh et al (1997) and Walsh et al. (1999). Briefly, solutions of Aβ1-42 (0.5 mg/ml) will be incubated at room temperature for 2-3 days and centrifuged at 16,000×g for 10 min to remove large aggregates. The supernatant will be fractionated by size-exclusion chromatography, using a Superdex 75 column, eluting the peaks with 70 mM NaCl and 5 mM Tris, pH 7.4. This procedure yields a symmetric peak in the void volume of the column which contains protofibrils and a peak of soluble oligomers in the included volume (Walsh et al., 1997).

Isolation of brain Aβ oligomers. To remove large amyloid plaques and partially concentrate Aβ oligomers, the brain tissue can be processed following protocols previously described by Kuo et al. (1996) and Permanne et al. (1997). Grey matter will be dissected free of vessels. The material will be homogenized in a glass homogenizer in 4 volumes (wt/vol) of TBS buffer containing protease inhibitors (Complete, Boehringer-Mannheim) and subjected to ultracentrifugation (100,000 g, 60 mins). The resulting supernatant contains Aβ oligomers.

### Example 2

### Isolation of Soluble Seed-Free (SF) Aβ

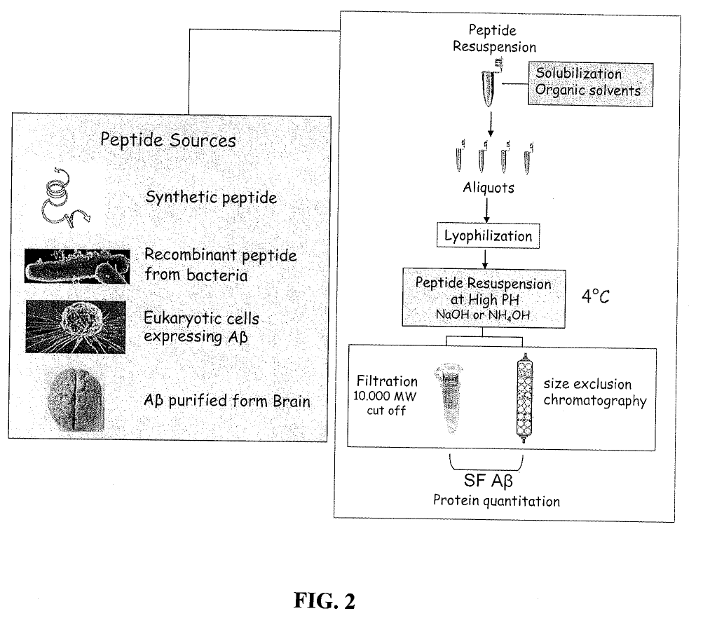

SF fractions of Aβ represent the substrate for the amplification reaction. FIG. 2 shows a schematic representation of the methodology for the preparation of SF Aβ. The first step is to dissolve the Aβ powder into an appropriate solvent to induce as much as possible the disassembly of preformed aggregates. Adequate solvents include various concentrations of sodium hydroxide, hexafluoroisopropanol, trifluoroacetic acid, dimethylsulfoxide, acetonitrile, guanidine hydrochloride, urea, formic acid, hydrochloride acid, ammonium hydroxide, trifluoroethanol, etc. The protein is dissolved in this buffer and incubated with agitation for 30 min and lyophilized. The lyophilized powder is resuspended in either 10 mM sodium or ammonium hydroxide, pH 12. The solution is passed through a size exclusion chromatography and the peak corresponding to a molecular weight between 4-12 KDa corresponds to the SF Aβ. Alternatively, instead of size exclusion chromatography, the samples could be filtrated through a 10 KDa cutoff filter and the material collected consists of SF Aβ. Protein concentration is determined by amino acid analysis or the BCA kit following manufacturer specifications. Samples are stored lyophilized at −80° C.

### Example 3

### Exemplary PMCA for TSE Diagnosis

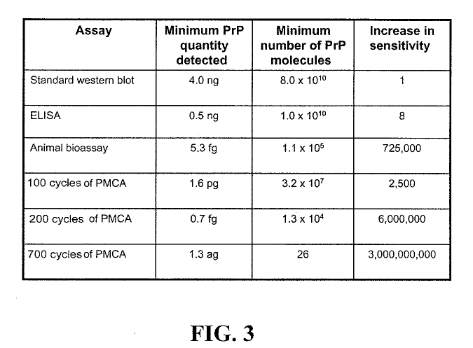

One of the Protein Misfolding Disorders in which the PMCA technology has been extensively studied are the transmissible spongiform encephalopathies (TSE) also known as prion diseases. The hallmark event in these diseases is the transformation of the normal prion protein (PrP^{C}) into a misfolded and toxic abnormal protein (PrP^{Sc}). A dramatic amplification of the PrP^{Sc} signal was reported by subjecting minute quantities of hamster PrP^{Sc} to PMCA in the presence of a large excess of PrP^{C} (Saborio et al., 2001). In addition, the inventors have demonstrated a clear increase in sensitivity for PrP^{Sc} detection by western blot, and an exponential relationship between the intensity of the PrP^{Sc} signal and the number of amplification cycles. More recently, the inventors have been able to automate the technology and apply it to replicate the misfolded protein from diverse species (Soto et al., 2005). The newly generated protein exhibits the same biochemical, biological, and structural properties as brain-derived PrP^{Sc} and strikingly it is infectious to wild-type animals, producing a disease with characteristics that are identical to the illness produced by brain-isolated misfolded proteins (Castilla et al., 2005b). FIG. 3 summarizes an experimental comparison of the efficiency of detection of various procedures with different number of PMCA cycles. The efficiency of misfolded protein amplification has been dramatically increased (more than 3 billion folds over standard tests) to the point that an estimated amount of 26 molecules of monomeric protein can be detected, which represents the equivalent to 1 unit of oligomeric PrP^{Sc}. This extremely high sensitivity enables detection of PrP^{Sc} with a very high sensitivity and specificity in blood of hamsters experimentally infected with scrapie both in the clinical phase (Castilla et al., 2005a) and during most of the pre-symptomatic period (Saa et al., 2006).

### Example 4

### Amplification of Aβ Aggregation

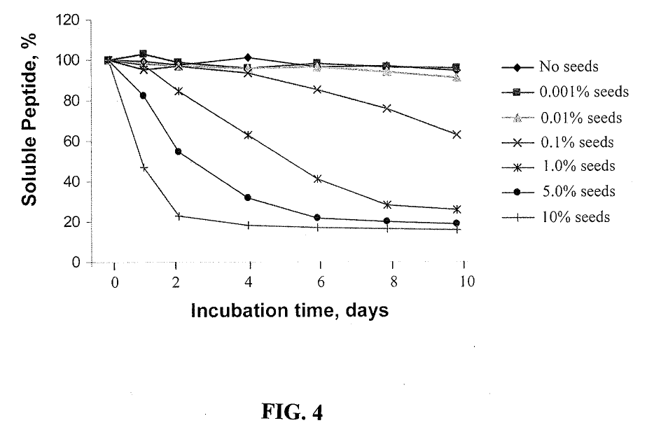

In implementing PMCA for Aβ aggregation, proof-of-concept studies have been performed using in vitro prepared seeds. Aβ seeds have been produced by incubating high concentrations of Aβ peptides to form fibrillar aggregates, followed by sonication to cut down the fibrils into smaller polymers. Alternatively, soluble oligomers and protofibrils can be produced and/or purified using the protocol described above. Samples containing low concentrations of “seeds-free” (SF) Aβ, i.e., Aβ substrate, will be incubated in the absence or in the presence of different quantities of Aβ seeds during various times and under various conditions. Low concentrations of SF Aβ42 (500 nM in 100 μl of 0.1 M sodium phosphate, pH 7.5) were incubated for several days at 37° C. alone or in the presence of the following concentrations of preformed seeds: 0.001%, 0.01%, 0.1%, 1%, 5% and 10%. Seed concentrations are expressed as a percentage of the soluble peptide in the solution. Seeds were prepared by incubating Aβ42 (50 μM) during 5 days at 37° C. and processed as described herein. Percentage of the SF peptide remaining soluble after different times was determined by sedimentation assays using a high sensitivity ELISA assay. As shown in FIG. 4, diluted low-molecular weight Aβ solution did not aggregate spontaneously under these experimental conditions during the time span in which this study was done. Lag phase is calculated to be larger than 7 days under these conditions. Addition of 0.1%, 1%, 5% and 10% of synthetic seeds induced aggregation of SF Aβ42 to an extent and with a kinetic dependent upon the quantity of seeds added (FIG. 4). This result suggests that the inventors can currently detect up to 0.5 nM or approximately 0.25 ng of oligomeric Aβ peptide by its capability to nucleate aggregation of soluble Aβ. Reduction of the quantity of Aβ seeds detectable by performing cycles of incubation/sonication will enable detecting aggregation triggered by lower concentrations of seeds through PMCA cycling (data not shown).

### Example 5

### Optimization of Amplification of Aβ Misfolding and Aggregation In Vitro

Several variables are evaluated to identify the conditions in which the highest sensitivity and reproducibility for cyclic amplification of Aβ misfolding and aggregation is obtained. These variables include peptide concentration, type of synthetic Aβ peptide (Aβ40 or Aβ42), time of incubation, shaking speed, sonication power, and temperature. The extent in which the soluble SF Aβ peptides aggregate under each condition is evaluated by a sedimentation assay in which the quantity of peptide remaining in solution will be measured by a high-sensitivity and high throughput sandwich ELISA assay. In some studies, the amyloid nature of the aggregated peptide is characterized by a variety of standard protocols including fluorometric thioflavine T assay, Congo red binding assay and electron microscopy. Controls will include incubation of seeds alone or addition of seeds to non-aggregating Aβ peptides with reverse sequence (Aβ42-1 or Aβ40-1). The methods are designed to detect specifically and reproducibly as little as 0.1-1.0 μM of Aβ oligomers, which corresponds to around 0.01-0.1% of total Aβ present in CSF or plasma (Andreasen et al., 1999).

After conditions are optimized for detection of small quantities of oligomers by seeded cyclic amplification of Aβ misfolding and aggregation, the inventors will evaluate the use of endogenous brain extracted Aβ oligomers to replace synthetically produced Aβ seeds. For these studies postmortem frozen brain tissue from AD patients and from single and double transgenic mice will be used as a source of brain Aβ oligomers, which will be partially purified as previously described (Permanne et al., 1997). Controls are done with samples obtained from normal brain homogenates from young and old individuals. These studies will test the unspecific seeding effect of other factors present in the brain homogenate.

### Example 6

### Identification of Aβ Misfolded Oligomers in AD Biological Fluids

CSF and blood plasma samples from normal young individuals are spiked with preformed synthetic Aβ seeds or with brain extracted seeds as described above. The aim of these studies is the development of conditions to obtain similar levels of detection of Aβ oligomers in biological fluids as in buffer. The study design is the same as before, i.e., samples containing minute quantities of Aβ oligomers are used to seed the cyclic amplification of diluted solutions of Aβ monomers. If the high concentrations of plasma proteins interfere with amplification or detection, the samples are first subjected to a cleaning step to remove the bulk of plasma proteins. For this purpose the inventors use immunoprecipitation with anti-Aβ antibodies or standard biochemical procedures to remove albumin and lipoprotein particles.

When conditions to detect oligomeric Aβ seeds in spiked samples are optimized, the inventors will detect putative Aβ oligomers in blood and CSF samples of transgenic animal models and human beings diagnosed or suspect of having AD. As controls, samples from age-matched non-transgenic mice and from normal people of different ages are used. For these studies the inventors use samples already available in the lab that were collected from collaborators and from a local patient population.

### Example 7

### Evaluation of the Sensitivity and Specificity of Aβ Oligomers Detection

The inventors will study the sensitivity and specificity of Aβ oligomer detection in biological fluids using a large number of samples from people diagnosed with AD and normal controls (both age-matched and young individuals). As part of the Mitchell Center for Alzheimer's disease research the inventors have an Alzheimer's clinic that is following up more than 100 patients at diverse stages of AD. Samples will be collected from these people as well as age-matched controls (from our geriatric clinic) and young individuals. For this study at least 100 samples of blood and CSF from each group will be used.

To further study specificity, the inventors will use samples from people affected by other neurological conditions, including Parkinson's, Huntington's disease, Creutzfeldt-Jakob disease, stroke, vascular dementia, amyotrophic lateral sclerosis (ALS) and Pick disease. These samples will be available from patients in Neurology and Geriatric clinics as well as from tissue and fluids banks.

A longitudinal study in AD transgenic mice will be performed to evaluate the earliest time in which Aβ oligomers can be detected in biological fluids. For these experiments CSF and blood samples are taken weekly from a group of control and single or double transgenic mice and subjected to PMCA detection of Aβ oligomers. Also, the inventors will evaluate the presence of Aβ oligomers in human populations at a high risk to develop AD, including non-symptomatic APP or presenilin mutant carriers and people with mild cognitive impairment.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. Aspects of one embodiment may be applied to other embodiments and vice versa. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,196,265
U.S. Pat. No. 5,688,648
U.S. Pat. No. 5,863,727
U.S. Pat. No. 6,008,373
U.S. Pat. No. 6,150,107
U.S. Pat. No. 6,855,503
WO 01/19841
Andreasen et al., Arch. Neurol., 56:673-680, 1999.
Bitan et al., J. Biol. Chem., 270:3063-3067, 1995.
Bucciantini et al., Nature, 416:507-511, 2002.
Castilla et al., Cell, 121:195-206, 2005a.
Castilla et al., Nat. Med., 11:982-985, 2005b.
Gong et al., Proc. Natl. Acad. Sci. USA, 100:10417-10422, 2003.
Hampel et al., J. Neural Transm., 111:247-272, 2004.
Hardy and Selkoe, Science, 297:353-356, 2002.
Harlow and Lane, “Antibodies: A Laboratory Manual,” Cold Spring Harbor Laboratory, 1988.
Harper and Lansbury, Annu. Rev. Biochem., 66:385-407, 1997.
Jarrett et al., Ann. NY Acad. Sci., 695:144-148, 1993.
Klunk et al., Ann. Neurol., 55:306-319, 2004.
Klunk et al., Methods Enzymol, 309:285-305, 1999.
Kung et al., Mol. Imaging. Biol., 5, 418-426, 2003.
Kuo et al., J. Biol. Chem., 271:4077-4081, 1996.
Lambert et al., Proc. Natl. Acad. Sci. USA, 95:6448-6453, 1998.
Levine, Neurobiol. Aging, 16:755-764, 1995.
Levine, Protein Sci., 2(3):404-410, 1993.
Mann et al., Neuropathol. Appl. Neurobiol., 16:17-25, 1990.
Mann, Ann. Med., 21:133-136, 1989.
Naiki et al., Anal. Biochem., 177(2):244-249, 1989.
Nestor et al., Nat. Med., 10 Suppl, S34-S41, 2004.
Permanne et al., Biochem. Biophys. Res. Commun., 240:715-720, 1997.
Pitschke et al, Nature Med., 4:832-834, 1998.
Saborio et al., Nature, 411:810-813, 2001.
Scherzinger et al., Proc. Natl. Acad. Sci. USA, 96:4604-4609, 1999.
Scott et al., Proc. Natl. Acad. Sci. USA, 96:15137-15142, 1999.
Selkoe and Schenk, Annu. Rev. Pharmacol. Toxicol., 43:545-584, 2003.
Selkoe, Ann. Intern. Med., 140:627-638, 2004.
Selkoe, Ann. NY Acad. Sci., 924:17-25, 2000.
Soto et al., FEBS Lett., 579:638-642, 2005.
Soto et al., J. Biol. Chem., 270(7):3063-3067, 2005b.
Soto et al., J. Clin. Microbiol., 36(1):58-63, 1998.
Soto et al., Neurosci. Lett., 200(2):105-108, 1005a.
Soto et al., Trends Neurosci., 25:390-394, 2002.
Soto, FEBS Lett., 498:204-207, 2001
Soto, Mol. Med. Today, 5:343-350, 1999.
Soto, Nat. Rev. Neurosci., 4:49-60, 2003.
Soto, Nature Rev. Microbiol., 2:809-819, 2004.
Saa et al., Science., 313(5783):92-94, 2006.
Teplow, Amyloid, 5:121-142, 1998.
Walsh and Selkoe, Protein Pept. Lett., 11:213-228, 2004.
Walsh et al., J. Biol. Chem., 272:22364-22372, 1997.
Walsh et al., J. Biol. Chem., 274:25945-25952, 1999.
Wood et al., J. Biol. Chem., 274:19509-19512, 1999.

1. A method for detecting a misfolded amyloid β (Aβ) protein in a sample comprising:
(a) mixing a sample obtained from a asymptomatic subject with an appropriate seed-free (SF) substrate amyloid β (Aβ) protein to make a reaction mix;
(b) incubating the reaction mix to enable conversion of the substrate amyloid β (Aβ) protein into the misfolded form; and
(c) detecting misfolding of the substrate amyloid β (Aβ) protein in the reaction mix.

2. The method of claim 1, having a sensitivity for detection of misfolded oligomeric Aβ ranging from 0.1 fentograms to 1 nanograms

3.-4. (canceled)

5. The method of claim 4, wherein the sample is from brain or a peripheral organ.

6. (canceled)

7. The method of claim 5, wherein the peripheral organ is blood, tears, urine, saliva, cerebrospinal fluid, peripheral nerves, skin, muscles, or lymphoid organs.

8.-9. (canceled)

10. The method of claim 1, wherein the substrate protein is a lysate.

11. The method of claim 10, wherein the lysate is a cell lysate or a brain homogenate.

12. (canceled)

13. The method of claim 11, wherein the brain homogenate is a mammalian brain homogenate.

14. The method of claim 13, wherein the brain homogenate is a human brain homogenate.

15. The method of claim 11, wherein the brain homogenate is a transgenic animal brain homogenate.

16. The method of claim 15, wherein the transgenic animal is a mouse.

17. (canceled)

18. The method of claim 1, wherein the sample is incubated at about 25° to 50° C.

19. The method of claim 1, wherein the sample is incubated for about 1 minute to about 10 hours.

20. (canceled)

21. The method of claim 1, wherein the reaction mixture further comprises a metal or a metal chelator.

22. The method of claim 21, wherein the metal chelator is EDTA.

23. The method of claim 1, wherein the misfolded protein is detected by a Western blot assay, an ELISA, a thioflavine T binding assay, a congo red binding assay, a sedimentation assay, an electron microscopic assessment, a spectroscopic assay, or a combination thereof.

24. A method for detecting a misfolded amyloid β (Aβ) protein in a sample comprising:
(a) mixing a sample from a subject that is asymptomatic for Alzheimer's disease with a substrate SF amyloid β (Aβ) protein to make a reaction mix;
(b) performing a cyclic amplification comprising;
(i) incubating the reaction mix;
(ii) disrupting the reaction mix;
(iii) repeating steps (i) and (ii) one or more times;
(c) detecting misfolded substrate amyloid β (Aβ) protein.

25.-46. (canceled)

47. The method of claim 24, wherein disrupting the sample is by sonication.

48. The method of claim 47, wherein the sonicator is programmable for automated operation.

49. The method of claim 47, wherein the sample does not directly contact the sonicator.

50. (canceled)

51. The method of claim 24, wherein steps (b)(i) and (b)(ii) are repeated 1 to 500 times.

52. The method of claim 24, wherein step (b) is performed over a period of about three days.

53. The method of claim 24, further comprising performing serial cyclic amplification by removing a portion of the reaction mix and incubating it with additional substrate protein.

54. The method of claim 53, wherein serial cyclic amplification is perform at least 1, 2, 3, 4, 5, 6, 7, 8, 9, 10 up to 100 times.

55. A method for detecting a misfolded amyloid β protein in a sample comprising;
(a) mixing the sample with the SF fraction obtained from a recombinant amyloid β 1-40 or a recombinant amyloid beta 1-42 substrate protein to make a reaction mix;
(b) performing a primary cyclic amplification comprising;
(i) incubating the reaction mix;
(ii) disrupting the reaction mix;
(iii) repeating steps (i) and (ii) one or more times;
(c) performing a serial cyclic amplification comprising;
(i) removing a portion of the reaction mix and incubating it with additional substrate protein;
(ii) repeating step (b);
(d) detecting misfolded protein in the reaction mix.

56.-57. (canceled)

58. A method to diagnose Alzheimer's disease in an asymptomatic human comprising detecting the presence of a misfolded protein in a sample from a patient suspected of having or at risk of having Alzheimer's disease by the method comprising:
(a) mixing the sample with a SF substrate amyloid β (Aβ) protein to make a reaction mix;
(b) performing a cyclic amplification comprising;
(i) incubating the reaction mix;
(ii) disrupting the reaction mix;
(iii) repeating steps (i) and (ii) one or more times;
(c) detecting misfolded substrate amyloid β (Aβ) protein.

59.-61. (canceled)
